(19) United States
(12) Patent Application Publication
(10) Pub. No.: US 2024/0189330 A1
(43) Pub. Date: Jun. 13, 2024

(54) Compositions and Methods for Inhibiting and Treating Viral Infections

(71) Applicant: The Regents of the University of California, Oakland, CA (US)

(72) Inventor: Theodoros Kelesidis, Dallas, TX (US)

(21) Appl. No.: 18/551,428

(22) PCT Filed: Mar. 23, 2022
(86) PCT No.: PCT/US2022/021511

§ 371 Date: Sep. 20, 2023

Related U.S. Application Data
(60) Provisional application No. 63/166,207, filed on Mar. 25, 2021.

(51) Int. Cl. A61K 31/66; A61K 31/225; A61P 31/14; A61P 31/16

(52) U.S. Cl. CPC A61K 31/66; A61K 31/225; A61P 31/14; A61P 31/16

(57) ABSTRACT

Disclosed herein are methods of using (a) one or more mitochondrial targeted antioxidants, (b) one or more Nrf2 agonists, and (c) combinations of one or more mitochondrial targeted antioxidants and one or more Nrf2 agonists, to prevent, inhibit, and/or treat infections and symptoms caused by infection by a virus. such as those belonging to the Orthomyxoviridae family, the Picornaviridae family, and the Pneumoviridae family of viruses.

### CROSS-REFERENCE TO RELATED APPLICATIONS

This application claims the benefit of U.S. Patent Application No. 63/166,207, filed Mar. 25, 2021, which is herein incorporated by reference in its entirety.

### BACKGROUND OF THE INVENTION

### 1. Field of the Invention

The field of the invention generally relates to compositions and methods for inhibiting and/or treating infections by viruses, e.g., viruses belonging to the Orthomyxoviridae family, the Picornaviridae family, and the Pneumoviridae family of viruses.

### 2. Description of the Related Art

The COVID-19 pandemic is evidence that the development of vaccines and antivirals is challenging because viruses such as SARS-COV-2 have high mutation rates. The pandemic also brought to light that many vaccines such as the COVID vaccines have suboptimal efficacy, especially in immunocompromised patients and against divergent strains. Thus, a need exists for a therapeutic that may be administered for long periods of time, e.g., chronic administration, as a pre- or post-exposure prophylactic and/or as an antiviral post-infection that does not depend on learned immunity.

### SUMMARY OF THE INVENTION

In some embodiments, the present invention is directed to methods of preventing, inhibiting, or reducing (a) infection by, (b) an inflammatory response caused by infection by, and/or (c) apoptosis caused by infection by a virus in a cell or a subject, which comprises, consists essentially of, or consists of administering one or more mitochondrial targeted antioxidants to the cell or the subject, wherein the virus is not a respiratory syncytial virus (RSV) or a coronavirus. In some embodiments, the present invention is directed to methods treating a subject for a viral disease caused by infection by a virus, which comprises, consists essentially of, or consists of administering one or more mitochondrial targeted antioxidants to the subject, wherein the virus is not a respiratory syncytial virus (RSV) or a coronavirus. In some embodiments, the present invention is directed to methods of preventing, inhibiting, or reducing infection by a virus in a subject, which comprises, consists essentially of, or consists of providing a plasma concentration of about 2 ng/ml or higher of one or more mitochondrial targeted antioxidants, wherein the virus is not a respiratory syncytial virus (RSV) or a coronavirus. In some embodiments, the one or more mitochondrial targeted antioxidants is selected from the group consisting of 10-(4,5-dimethoxy-2-methyl-3,6-dioxo-1,4-cyclohexadienyl) decyl triphenylphosphonium and salts thereof, 10-(4,5-dimethoxy-2-methyl-3,6-dihydroxy-1,4-cyclohexadienyl) decyl triphenylphosphonium and salts thereof, SkQ1, Elamipretide, and Mito-TEMPO. In some embodiments, the one or more mitochondrial targeted antioxidants is Mito-MES (i.e., mitoquinone mesylate and/or mitoquinol mesylate). In some embodiments, about 0.05 mg/kg to about 15 mg/kg, preferably about 0.2 mg/kg to about 1.5 mg/kg, or more preferably about 0.3 mg/kg to about 0.7 mg/kg of Mito-MES per weight of the subject is administered to the subject. In some embodiments, about 1-1000 mg, 5-100 mg, 10-80 mg, or 20-40 mg, preferably about 20 mg of Mito-MES is administered to the subject. In some embodiments, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg of Mito-MES is administered to the subject. In some embodiments, the one or more mitochondrial targeted antioxidants is administered daily. In some embodiments, the one or more mitochondrial targeted antioxidants is administered orally, subcutaneously, or intravenously, preferably orally. In some embodiments, the administration of the one or more mitochondrial targeted antioxidants is before, during, and/or after the subject was exposed or likely exposed to the virus. In some embodiments, the administration of the one or more mitochondrial targeted antioxidants occurs for at least 1, 2, 3, 4, 5, 6, 7, 8, 9, 10, 11, 12, 13, or 14 days, after the exposure or likely exposure to the virus. In some embodiments, the administration of the one or more mitochondrial targeted antioxidants occurs for at least 1-10 days after the exposure or likely exposure to the virus. In some embodiments, the virus belongs to the Orthomyxoviridae family, preferably the virus is an Alphainfluenzavirus, a Betainfluenzavirus, a Gammainfluenzavirus, a Deltainfluenzavirus, an Isavirus, a Quaranjavirus, or a Thogotovirus, more preferably, the virus is an Influenza A virus, an Influenza B virus, an Influenza C virus, or an Influenza D virus, even more preferably the virus is an Influenza A virus of subtype H1N1, H2N2, H3N2, H5N1, H7N9, H7N7, H1N2, H9N2, H7N2, H7N3, H5N2, H10N7, H10N3, or H5N8. In some embodiments, the virus is an H1N1 Influenza A virus.

In some embodiments, the present invention is directed to the use of one or more mitochondrial targeted antioxidants (a) as an antiviral against a virus, or (b) to prevent, inhibit, or reduce apoptosis caused by infection by the virus, wherein the virus is not a respiratory syncytial virus (RSV) or a coronavirus. In some embodiments, the present invention is directed to one or more mitochondrial targeted antioxidants or a composition thereof for use (a) in the treatment of an infection by a virus, or (b) as a medicament in the treatment of an infection by the virus, wherein the virus is not a respiratory syncytial virus (RSV) or a coronavirus. In some embodiments, the one or more mitochondrial targeted antioxidants is selected from the group consisting of 10-(4,5-dimethoxy-2-methyl-3,6-dioxo-1,4-cyclohexadienyl) decyl triphenylphosphonium and salts thereof, 10-(4,5-dimethoxy-2-methyl-3,6-dihydroxy-1,4-cyclohexadienyl) decyl triphenylphosphonium and salts thereof, SkQ1, Elamipretide, and Mito-TEMPO. In some embodiments, the one or more mitochondrial targeted antioxidants is Mito-MES (i.e., mitoquinone mesylate and/or mitoquinol mesylate). In some embodiments, an amount of Mito-MES of about 1-1000 mg, 5-100 mg, 10-80 mg, or 20-40 mg, preferably about 20 mg is used or provided. In some embodiments, an amount of Mito-MES of 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg is used or provided. In some embodiments, the one or more mitochondrial targeted antioxidants is provided as a daily dose. In some embodiments, the one or more mitochondrial targeted antioxidants is provided as an oral, subcutaneous, or intravenous, preferably oral, formulation. In some embodiments, the use of the one or more mitochondrial targeted antioxidants is before, during, and/or after exposure or likely exposure to the virus. In some embodiments, the use of the one or more mitochondrial targeted antioxidants occurs for at least 1, 2, 3, 4, 5, 6, 7, 8, 9, 10, 11, 12, 13, or 14 days, after exposure or likely exposure to the virus. In some embodiments, the use of the one or more mitochondrial targeted antioxidants occurs for at least 1-10 days after exposure or likely exposure to virus. In some embodiments, the virus belongs to the Orthomyxoviridae family, preferably the virus is an Alphainfluenzavirus, a Betainfluenzavirus, a Gammainfluenzavirus, a Deltainfluenzavirus, an Isavirus, a Quaranjavirus, or a Thogotovirus, more preferably, the virus is an Influenza A virus, an Influenza B virus, an Influenza C virus, or an Influenza D virus, even more preferably the virus is an Influenza A virus of subtype H1N1, H2N2, H3N2, H5N1, H7N9, H7N7, H1N2, H9N2, H7N2, H7N3, H5N2, H10N7, H10N3, or H5N8. In some embodiments, the virus is an H1N1 Influenza A virus.

In some embodiments, the present invention is directed to methods of preventing, inhibiting, or reducing (a) infection by, (b) an inflammatory response caused by infection by, and/or (c) apoptosis caused by infection by a virus in a cell or a subject, which comprises, consists essentially of, or consists of administering one or more Nrf2 agonists to the cell or the subject, wherein the virus is not a coronavirus. In some embodiments, the present invention is directed to methods treating a subject for a viral disease caused by infection by a virus, which comprises, consists essentially of, or consists of administering one or more Nrf2 agonists to the subject, wherein the virus is not a coronavirus. In some embodiments, the one or more Nrf2 agonists is selected from Antcin C, Baicalein, Butein and phloretin, Carthamus red, Curcumin, Diallyl disulfide, Ellagic acid, Gastrodin, Ginsenoside Rg1, Ginsenoside Rg3, Glycyrrhetinic acid, Hesperidin, Isoorientin, Linalool, Lucidone, Lutein, Lycopene, Mangiferin, Naringenin, Oleanolic acid, Oroxylin A, Oxyresveratrol, Paeoniflorin, Puerarin, Quercetin, Resveratrol, S-Allylcysteine, Salvianolic acid B, Sauchinone, Schisandrin B, Sulforaphane, Tungtungmadic acid, Withaferin A, Alpha-lipoic acid, and Dimethyl fumarate (DMF). In some embodiments, the one or more Nrf2 agonists is dimethyl fumarate (DMF). In some embodiments, about 0.02-8.0 mg/kg, about 0.15-8.0 mg/kg, about 4.0-8.0 mg/kg, about 0.01-4.0 mg/kg, about 0.1-4.0 mg/kg, or about 2.0-4.0 mg/kg of DMF per weight of the subject is administered to the subject. In some embodiments, about 1-480 mg, about 10-480 mg, about 240-480 mg, about 0.5-240 mg, about 5-240 mg, or about 120-240 mg of DMF is administered to the subject. In some embodiments, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, or 480 mg of DMF is administered to the subject. In some embodiments, the one or more Nrf2 agonists is administered daily. In some embodiments, the one or more Nrf2 agonists is administered orally, subcutaneously, or intravenously, preferably orally. In some embodiments, the administration of the one or more Nrf2 agonists is before, during, and/or after the subject was exposed or likely exposed to the virus. In some embodiments, the administration of the one or more Nrf2 agonists occurs for at least 1, 2, 3, 4, 5, 6, 7, 8, 9, 10, 11, 12, 13, or 14 days, after the exposure or likely exposure to the virus. In some embodiments, the administration of the one or more Nrf2 agonists occurs for at least 1-10 days after the exposure or likely exposure to the virus. In some embodiments, the virus belongs to the Orthomyxoviridae family, preferably the virus is an Alphainfluenzavirus, a Betainfluenzavirus, a Gammainfluenzavirus, a Deltainfluenzavirus, an Isavirus, a Quaranjavirus, or a Thogotovirus, more preferably, the virus is an Influenza A virus, an Influenza B virus, an Influenza C virus, or an Influenza D virus, even more preferably the virus is an Influenza A virus of subtype H1N1, H2N2, H3N2, H5N1, H7N9, H7N7, H1N2, H9N2, H7N2, H7N3, H5N2, H10N7, H10N3, or H5N8. In some embodiments, the virus is an H1N1 Influenza A virus.

In some embodiments, the present invention is directed to the use of one or more Nrf2 agonists (a) as an antiviral against a virus, or (b) to prevent, inhibit, or reduce apoptosis caused by infection by the virus, wherein the virus is not a coronavirus. In some embodiments, the present invention is directed to one or more Nrf2 agonists or a composition thereof for use (a) in the treatment of an infection by a virus, or (b) as a medicament in the treatment of an infection by the virus, wherein the virus is not a coronavirus. In some embodiments, the one or more Nrf2 agonists is selected from Antcin C, Baicalein, Butein and phloretin, Carthamus red, Curcumin, Diallyl disulfide, Ellagic acid, Gastrodin, Ginsenoside Rg1, Ginsenoside Rg3, Glycyrrhetinic acid, Hesperidin, Isoorientin, Linalool, Lucidone, Lutein, Lycopene, Mangiferin, Naringenin, Oleanolic acid, Oroxylin A, Oxyresveratrol, Paeoniflorin, Puerarin, Quercetin, Resveratrol, S-Allylcysteine, Salvianolic acid B, Sauchinone, Schisandrin B, Sulforaphane, Tungtungmadic acid, Withaferin A, Alpha-lipoic acid, and Dimethyl fumarate (DMF). In some embodiments, the one or more Nrf2 agonists is dimethyl fumarate (DMF). In some embodiments, an amount of DMF of about 1-480 mg, about 10-480 mg, about 240-480 mg, about 0.5-240 mg, about 5-240 mg, or about 120-240 mg is used or provided. In some embodiments, an amount of DMF of 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, or 480 mg is used or provided. In some embodiments, the one or more Nrf2 agonists is provided as a daily dose. In some embodiments, the one or more Nrf2 agonists is provided as an oral, subcutaneous, or intravenous, preferably oral, formulation. In some embodiments, the use of the one or more Nrf2 agonists is before, during, and/or after exposure or likely exposure to the virus. In some embodiments, the use of the one or more Nrf2 agonists occurs for at least 1, 2, 3, 4, 5, 6, 7, 8, 9, 10, 11, 12, 13, or 14 days, after exposure or likely exposure to the virus. In some embodiments, the use of the one or more Nrf2 agonists occurs for at least 1-10 days after exposure or likely exposure to virus. In some embodiments, the virus belongs to the Orthomyxoviridae family, preferably the virus is an Alphainfluenzavirus, a Betainfluenzavirus, a Gammainfluenzavirus, a Deltainfluenzavirus, an Isavirus, a Quaranjavirus, or a Thogotovirus, more preferably, the virus is an Influenza A virus, an Influenza B virus, an Influenza C virus, or an Influenza D virus, even more preferably the virus is an Influenza A virus of subtype H1N1, H2N2, H3N2, H5N1, H7N9, H7N7, H1N2, H9N2, H7N2, H7N3, H5N2, H10N7, H10N3, or H5N8. In some embodiments, the virus is an H1N1 Influenza A virus.

In some embodiments, the present invention is directed to methods of preventing, inhibiting, or reducing (a) infection by, (b) an inflammatory response caused by infection by, and/or (c) apoptosis caused by infection by a virus in a cell or a subject, which comprises, consists essentially of, or consists of administering a combination of one or more mitochondrial targeted antioxidants+one or more Nrf2 agonists to the cell or the subject, wherein the virus is not a coronavirus. In some embodiments, the present invention is directed to methods treating a subject for a viral disease caused by infection by a virus, which comprises, consists essentially of, or consists of administering a combination of one or more mitochondrial targeted antioxidants+one or more Nrf2 agonists to the subject, wherein the virus is not a coronavirus. In some embodiments, the present invention is directed to methods of preventing, inhibiting, or reducing infection by a virus in a subject, which comprises, consists essentially of, or consists of providing a plasma concentration of about 2 ng/ml or higher of one or more mitochondrial targeted antioxidants in the subject in combination with administering one or more Nrf2 agonists to the subject, wherein the virus is not a coronavirus. In some embodiments, the virus is not a respiratory syncytial virus (RSV). In some embodiments, the one or more mitochondrial targeted antioxidants is selected from the group consisting of 10-(4,5-dimethoxy-2-methyl-3,6-dioxo-1,4-cyclohexadienyl) decyl triphenylphosphonium and salts thereof, 10-(4,5-dimethoxy-2-methyl-3,6-dihydroxy-1,4-cyclohexadienyl) decyl triphenylphosphonium and salts thereof, SkQ1, Elamipretide, and Mito-TEMPO. In some embodiments, the one or more mitochondrial targeted antioxidants is Mito-MES (i.e., mitoquinone mesylate and/or mitoquinol mesylate). In some embodiments, about 0.05 mg/kg to about 15 mg/kg, preferably about 0.2 mg/kg to about 1.5 mg/kg, or more preferably about 0.3 mg/kg to about 0.7 mg/kg of Mito-MES per weight of the subject is administered to the subject. In some embodiments, about 1-1000 mg, 5-100 mg, 10-80 mg, or 20-40 mg, preferably about 20 mg of Mito-MES is administered to the subject. In some embodiments, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg of Mito-MES is administered to the subject. In some embodiments, the one or more Nrf2 agonists is selected from Antcin C, Baicalein, Butein and phloretin, Carthamus red, Curcumin, Diallyl disulfide, Ellagic acid, Gastrodin, Ginsenoside Rg1, Ginsenoside Rg3, Glycyrrhetinic acid, Hesperidin, Isoorientin, Linalool, Lucidone, Lutein, Lycopene, Mangiferin, Naringenin, Oleanolic acid, Oroxylin A, Oxyresveratrol, Paeoniflorin, Puerarin, Quercetin, Resveratrol, S-Allylcysteine, Salvianolic acid B, Sauchinone, Schisandrin B, Sulforaphane, Tungtungmadic acid, Withaferin A, Alpha-lipoic acid, and Dimethyl fumarate (DMF). In some embodiments, the one or more Nrf2 agonists is dimethyl fumarate (DMF). In some embodiments, about 0.02-8.0 mg/kg, about 0.15-8.0 mg/kg, about 4.0-8.0 mg/kg, about 0.01-4.0 mg/kg, about 0.1-4.0 mg/kg, or about 2.0-4.0 mg/kg of DMF per weight of the subject is administered to the subject. In some embodiments, about 1-480 mg, about 10-480 mg, about 240-480 mg, about 0.5-240 mg, about 5-240 mg, or about 120-240 mg of DMF is administered to the subject. In some embodiments, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, or 480 mg of DMF is administered to the subject. In some embodiments, the amount of the one or more mitochondrial targeted antioxidants to the amount of the one or more Nrf2 agonists is about 1:10. In some embodiments, the one or more mitochondrial targeted antioxidants and/or the one or more Nrf2 agonists is administered daily. In some embodiments, the one or more mitochondrial targeted antioxidants and/or the one or more Nrf2 agonists is administered orally, subcutaneously, or intravenously, preferably orally. In some embodiments, the administration of the one or more mitochondrial targeted antioxidants and/or the one or more Nrf2 agonists is before, during, and/or after the subject was exposed or likely exposed to the virus. In some embodiments, the administration of the one or more mitochondrial targeted antioxidants and/or the one or more Nrf2 agonists occurs for at least 1, 2, 3, 4, 5, 6, 7, 8, 9, 10, 11, 12, 13, or 14 days, after the exposure or likely exposure to the virus. In some embodiments, the administration of the one or more mitochondrial targeted antioxidants and/or the one or more Nrf2 agonists occurs for at least 1-10 days after the exposure or likely exposure to the virus. In some embodiments, the one or more mitochondrial targeted antioxidants and the one or more Nrf2 agonists are administered to a cell or a subject concurrently. In some embodiments, the one or more mitochondrial targeted antioxidants and the one or more Nrf2 agonists are administered to a cell or a subject consecutively or at different times or on different days. In some embodiments, the one or more mitochondrial targeted antioxidants and the one or more Nrf2 agonists are provided as separate compositions. In some embodiments, the one or more mitochondrial targeted antioxidants and the one or more Nrf2 agonists are provided together as a single composition. In some embodiments, the virus belongs to the Orthomyxoviridae family, preferably the virus is an Alphainfluenzavirus, a Betainfluenzavirus, a Gammainfluenzavirus, a Deltainfluenzavirus, an Isavirus, a Quaranjavirus, or a Thogotovirus, more preferably, the virus is an Influenza A virus, an Influenza B virus, an Influenza C virus, or an Influenza D virus, even more preferably the virus is an Influenza A virus of subtype H1N1, H2N2, H3N2, H5N1, H7N9, H7N7, H1N2, H9N2, H7N2, H7N3, H5N2, H10N7, H10N3, or H5N8. In some embodiments, the virus is an H1N1 Influenza A virus. In some embodiments, the virus belongs to the Picornaviridae family, preferably the virus is an Enterovirus (preferably a human Enterovirus), more preferably the virus is an Enterovirus A, an Enterovirus B, an Enterovirus C, an Enterovirus D, a Rhinovirus A, a Rhinovirus B, or a Rhinovirus C virus, even more preferably the virus is a Rhinovirus A, a Rhinovirus B, or a Rhinovirus C virus, and most preferably the virus is a Rhinovirus A virus such as human rhinovirus HRV16. In some embodiments, the virus belongs to the Pneumoviridae family, preferably the virus is a Metapneumovirus or a Orthopneumovirus, more preferably the virus is a human Metapneumovirus or a human Orthopneumovirus, even more preferably the virus is a human respiratory syncytial virus, and most preferably the virus is a human metapneumovirus (HMPV), a human respiratory syncytial virus A2 (HRSV-A2), or a human respiratory syncytial virus B1 (HRSV-B1).

In some embodiments, the present invention is directed to the use of a combination of one or more mitochondrial targeted antioxidants+one or more Nrf2 agonists (a) as an antiviral against a virus, or (b) to prevent, inhibit, or reduce apoptosis caused by infection by the virus, wherein the virus is not a coronavirus. In some embodiments, the present invention is directed to a combination of one or more mitochondrial targeted antioxidants+one or more Nrf2 agonists or a composition thereof for use (a) in the treatment of an infection by a virus, or (b) as a medicament in the treatment of an infection by the virus, wherein the virus is not a coronavirus. In some embodiments, the virus is not a respiratory syncytial virus (RSV). In some embodiments, the one or more mitochondrial targeted antioxidants is selected from the group consisting of 10-(4,5-dimethoxy-2-methyl-3,6-dioxo-1,4-cyclohexadienyl) decyl triphenylphosphonium and salts thereof, 10-(4,5-dimethoxy-2-methyl-3,6-dihydroxy-1,4-cyclohexadienyl) decyl triphenylphosphonium and salts thereof, SkQ1, Elamipretide, and Mito-TEMPO. In some embodiments, the one or more mitochondrial targeted antioxidants is Mito-MES (i.e., mitoquinone mesylate and/or mitoquinol mesylate). In some embodiments, about 1-1000 mg, 5-100 mg, 10-80 mg, or 20-40 mg, preferably about 20 mg of Mito-MES is used or provided. In some embodiments, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg of Mito-MES is used or provided. In some embodiments, the one or more Nrf2 agonists is selected from Antcin C, Baicalein, Butein and phloretin, Carthamus red, Curcumin, Diallyl disulfide, Ellagic acid, Gastrodin, Ginsenoside Rg1, Ginsenoside Rg3, Glycyrrhetinic acid, Hesperidin, Isoorientin, Linalool, Lucidone, Lutein, Lycopene, Mangiferin, Naringenin, Oleanolic acid, Oroxylin A, Oxyresveratrol, Paeoniflorin, Puerarin, Quercetin, Resveratrol, S-Allylcysteine, Salvianolic acid B, Sauchinone, Schisandrin B, Sulforaphane, Tungtungmadic acid, Withaferin A, Alpha-lipoic acid, and Dimethyl fumarate (DMF). In some embodiments, the one or more Nrf2 agonists is dimethyl fumarate (DMF). In some embodiments, about 1-480 mg, about 10-480 mg, about 240-480 mg, about 0.5-240 mg, about 5-240 mg, or about 120-240 mg of DMF is used or provided. In some embodiments, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, or 480 mg of DMF is used or provided. In some embodiments, the amount of the one or more mitochondrial targeted antioxidants to the amount of the one or more Nrf2 agonists is about 1:10. In some embodiments, the one or more mitochondrial targeted antioxidants and/or the one or more Nrf2 agonists is provided as a daily dose. In some embodiments, the one or more mitochondrial targeted antioxidants and/or the one or more Nrf2 agonists is provided as an oral, subcutaneous, or intravenous, preferably oral, formulation. In some embodiments, the use of the one or more mitochondrial targeted antioxidants and/or the one or more Nrf2 agonists is before, during, and/or after exposure or likely exposure to the virus. In some embodiments, the use of the one or more mitochondrial targeted antioxidants and/or the one or more Nrf2 agonists occurs for at least 1, 2, 3, 4, 5, 6, 7, 8, 9, 10, 11, 12, 13, or 14 days, after exposure or likely exposure to the virus. In some embodiments, the use of the one or more mitochondrial targeted antioxidants and/or the one or more Nrf2 agonists occurs for at least 1-10 days after exposure or likely exposure to virus. In some embodiments, the one or more mitochondrial targeted antioxidants and the one or more Nrf2 agonists are packaged together or formulated for concurrent administration. In some embodiments, the one or more mitochondrial targeted antioxidants and the one or more Nrf2 agonists are packaged or formulated to be administered consecutively or at different times or on different days. In some embodiments, the one or more mitochondrial targeted antioxidants and the one or more Nrf2 agonists are provided as separate compositions. In some embodiments, the one or more mitochondrial targeted antioxidants and the one or more Nrf2 agonists are provided together as a single composition. In some embodiments, the virus belongs to the Orthomyxoviridae family, preferably the virus is an Alphainfluenzavirus, a Betainfluenzavirus, a Gammainfluenzavirus, a Deltainfluenzavirus, an Isavirus, a Quaranjavirus, or a Thogotovirus, more preferably, the virus is an Influenza A virus, an Influenza B virus, an Influenza C virus, or an Influenza D virus, even more preferably the virus is an Influenza A virus of subtype H1N1, H2N2, H3N2, H5N1, H7N9, H7N7, H1N2, H9N2, H7N2, H7N3, H5N2, H10N7, H10N3, or H5N8. In some embodiments, the virus is an H1N1 Influenza A virus. In some embodiments, the virus belongs to the Picornaviridae family, preferably the virus is an Enterovirus (preferably a human Enterovirus), more preferably the virus is an Enterovirus A, an Enterovirus B, an Enterovirus C, an Enterovirus D, a Rhinovirus A, a Rhinovirus B, or a Rhinovirus C virus, even more preferably the virus is a Rhinovirus A, a Rhinovirus B, or a Rhinovirus C virus, and most preferably the virus is a Rhinovirus A virus such as human rhinovirus HRV16. In some embodiments, the virus belongs to the Pneumoviridae family, preferably the virus is a Metapneumovirus or a Orthopneumovirus, more preferably the virus is a human Metapneumovirus or a human Orthopneumovirus, even more preferably the virus is a human respiratory syncytial virus, and most preferably the virus is a human metapneumovirus (HMPV), a human respiratory syncytial virus A2 (HRSV-A2), or a human respiratory syncytial virus B1 (HRSV-B1).

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1: Viral infection promotes formation of Mito-ROS that are instigators of mitochondrial dysfunction and an interdependent cycle of epithelial and immune cell dysfunction as well as inflammation and Mito-MES inhibits Mito-ROS formation and the downstream inflammatory response caused by Mito-ROS.

FIG. 2: Mito-MES and DMF inhibit H1N1 replication in lung epithelial cells. A546 lung epithelial cells were treated with 10 μM of oseltamivir (antiviral positive control) or the indicated doses of Mito-MES, DMF, or DMSO vehicle control (Ctrl). Viral replication at 48 hrs post infection (hpi) by qPCR. For all experiments cells were infected with virus at an MOI of 0.5 and they were treated with drugs for 2 hrs before infection and throughout the experiment until 48 hpi. In all panels summary data are presented as mean±SEM. Unless otherwise stated, statistical comparison was done between the Ctrl and each shown experimental group by using Kruskal Wallis or two-tailed Mann-Whitney (*p<0.05, **p<0.01, ***p<0.001).

FIG. 3: Mito-MES inhibits H1N1 replication in human bronchial epithelial cells. Human bronchial airway epithelial cells (HBECs) were treated with 10 μM of oseltamivir (antiviral positive control) or the indicated doses of Mito-MES, or DMSO vehicle control (Ctrl). Viral replication at 48 hrs post infection (hpi) by qPCR. For all experiments cells were infected with virus at an MOI of 0.5 and they were treated with drugs for 2 hrs before infection and throughout the experiment until 48 hpi. In all panels summary data are presented as mean±SEM. Unless otherwise stated, statistical comparison was done between the Ctrl and each shown experimental group by using Kruskal Wallis or two-tailed Mann-Whitney (*p<0.05, **p<0.01, ***p<0.001).

FIG. 4: Mito-MES alone and DMF alone minimally inhibit HRV16 replication in lung epithelial cells, but the combination of Mito-MES+DMF synergistically inhibits viral replication. A546 lung epithelial cells were treated with 10 μM of oseltamivir (antiviral positive control) or the indicated doses of Mito-MES, DMF, or DMSO vehicle control (Ctrl). Viral replication at 48 hrs post infection (hpi) by qPCR. For all experiments cells were infected with virus at an MOI of 0.5 and they were treated with drugs for 2 hrs before infection and throughout the experiment until 48 hpi. In all panels summary data are presented as mean±SEM. Unless otherwise stated, statistical comparison was done between the Ctrl and each shown experimental group by using Kruskal Wallis or two-tailed Mann-Whitney (*p<0.05, **p<0.01, ***p<0.001).

FIG. 5: Mito-MES by itself minimally inhibits HRV16 replication in human bronchial epithelial cells. Human bronchial airway epithelial cells (HBECs) were treated with 10 μM of oseltamivir (antiviral positive control) or the indicated doses of Mito-MES, or DMSO vehicle control (Ctrl). Viral replication at 48 hrs post infection (hpi) by qPCR. For all experiments cells were infected with virus at an MOI of 0.5 and they were treated with drugs for 2 hrs before infection and throughout the experiment until 48 hpi. In all panels summary data are presented as mean±SEM. Unless otherwise stated, statistical comparison was done between the Ctrl and each shown experimental group by using Kruskal Wallis or two-tailed Mann-Whitney (*p<0.05, **p<0.01, ***p<0.001).

FIG. 6: Mito-MES and DMF synergistically inhibit RSV replication in lung epithelial cells. A546 lung epithelial cells were treated with the indicated doses of Mito-MES, DMF, or DMSO vehicle control (Ctrl). Viral replication at 48 hrs post infection (hpi) by qPCR. For all experiments cells were infected with virus at an MOI of 0.5 and they were treated with drugs for 2 hrs before infection and throughout the experiment until 48 hpi. In all panels summary data are presented as mean±SEM. Unless otherwise stated, statistical comparison was done between the Ctrl and each shown experimental group by using Kruskal Wallis or two-tailed Mann-Whitney (*p<0.05, **p<0.01, ***p<0.001).

### DETAILED DESCRIPTION OF THE INVENTION

WO 2022015570, which is herein incorporated by reference, discloses that mitochondrial targeted antioxidants, such as Mito-MES (i.e., mitoquinone mesylate and/or mitoquinol mesylate), exhibit antiviral and post-exposure prophylaxis activity against coronaviruses, such as SARS-COV-2. Mito-MES is also known to have antiviral activity against Respiratory Syncytial Virus (RSV).

Virus infects epithelial and immune cells and viral proteins induce several cellular changes such as formation of protein-lipid channels in ER/Golgi, double membrane vesicles (DMVs) and degradation of Mitochondrial Antiviral Signaling Protein (MAVS) and proteins important for mitochondrial dynamics (e.g., Drp1). Collectively these changes lead to altered Ca2+ signaling, centrosome-derived cellular trafficking and increased mitochondrial reactive oxygen species (Mito-ROS). Mito-ROS trigger several cellular changes that induce metabolic reprogramming and ultimately viral replication.

Because initial experimental data suggested that Mito-MES likely exerts its antiviral activity against SARS-COV-2 via inhibiting or reducing Mito-ROS generation and regulating the interferon pathway and inflammatory response caused by SARS-COV-2, it was hypothesized that mitochondrial targeted antioxidants, such as Mito-MES, could be used as an antiviral against a broad spectrum of viruses. This is schematically shown in FIG. 1.

However, as disclosed herein, while Mito-MES exhibits antiviral activity against viruses belonging to the Orthomyxoviridae family, mitoquinone mesylate by itself exhibits little antiviral activity against viruses belonging to the Picornaviridae family.

As disclosed herein, Mito-MES is used as a representative species of mitochondrial targeted antioxidants and dimethyl fumarate (DMF) is used as a representative species of Nrf2 agonists.

### Mito-MES Antiviral Activity Against Orthomyxoviridae Viruses

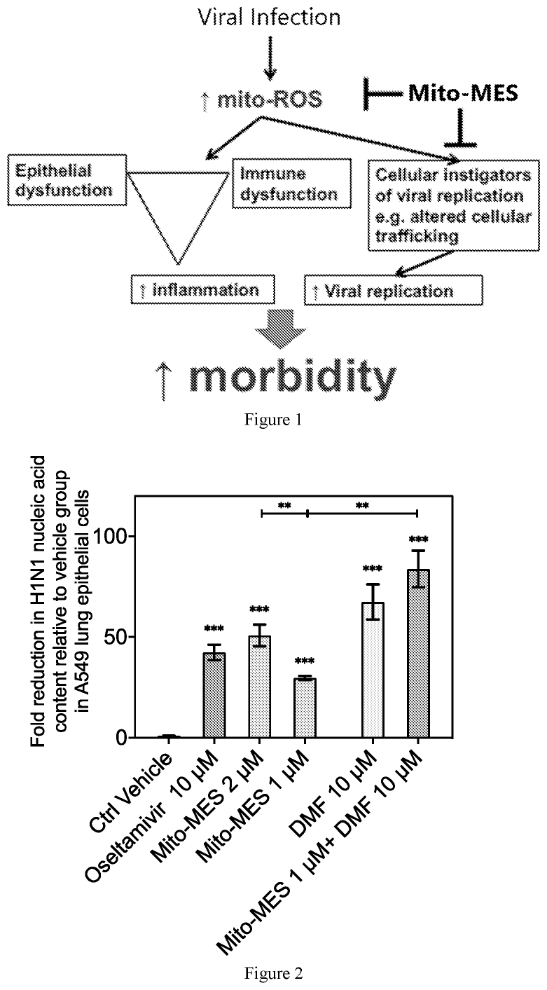

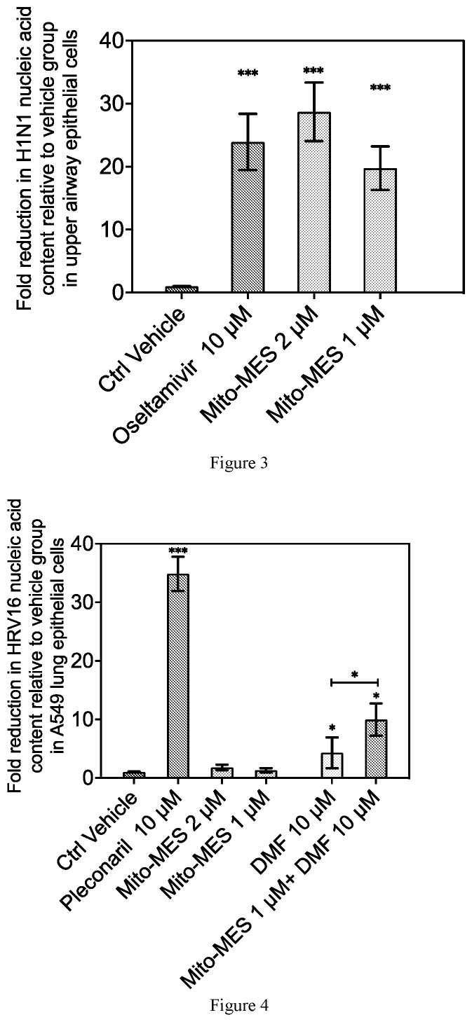

The “H1N1” virus (Influenza A Virus, subtype H1N1), was used as being representative of viruses belonging to the Orthomyxoviridae family of viruses. As shown in FIG. 2, Mito-MES and DMF, alone and in combination, inhibit the replication of viruses belonging to the Orthomyxoviridae family in lung epithelial cells. A546 lung epithelial cells were treated with 10 μM of oseltamivir (antiviral positive control), 2 μM and 1 μM of Mito-MES, 10 μM of DMF, 1 μM Mito-MES+10 μM DMF, or control vehicle (DMSO, negative control). Mito-MES also inhibits H1N1 replication in human bronchial epithelial cells. See FIG. 3. Human bronchial airway epithelial cells (HBECs) were treated with 10 μM of oseltamivir (antiviral positive control), 1 μM and 2 μM Mito-MES, or control vehicle (DMSO, negative control).

Therefore, in some embodiments, the present invention is directed to a method of preventing, inhibiting, or reducing infection by a virus belonging to the Orthomyxoviridae family of viruses in a cell or a subject, which comprises administering (a) one or more mitochondrial targeted antioxidants, and/or (b) one or more Nrf2 agonists to the cell or the subject.

In some embodiments, the virus is an Alphainfluenzavirus, a Betainfluenzavirus, a Gammainfluenzavirus, a Deltainfluenzavirus, an Isavirus, a Quaranjavirus, or a Thogotovirus. In some embodiments, the virus is an Alphainfluenzavirus, a Betainfluenzavirus, a Gammainfluenzavirus, or a Deltainfluenzavirus. In some embodiments, the virus is an Influenza A virus, an Influenza B virus, an Influenza C virus, or an Influenza D virus. In some embodiments, the subtype of the Influenza A virus is H1N1, H2N2, H3N2, H5N1, H7N9, H7N7, H1N2, H9N2, H7N2, H7N3, H5N2, H10N7, H10N3, or H5N8. In some embodiments, the Influenza A virus is an H1N1 virus.

### Mito-MES+DMF Synergistic Antiviral Activity

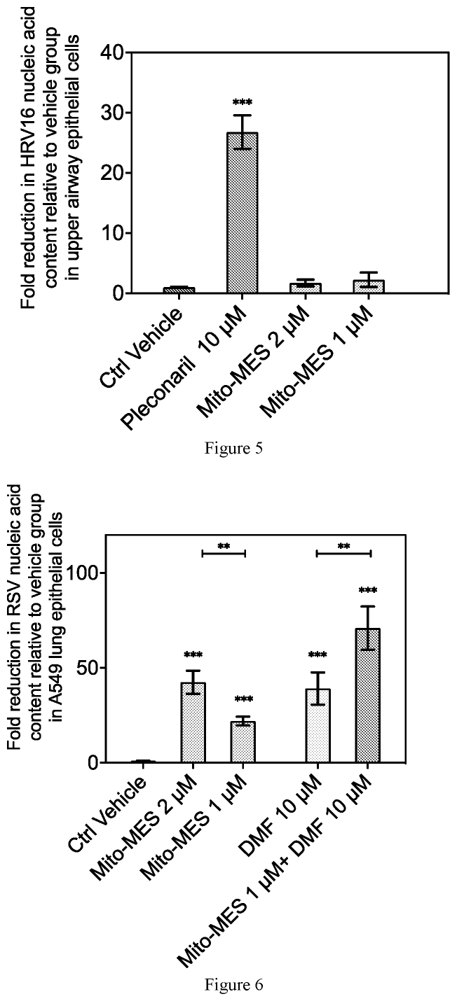

Human rhinovirus type 16 (HRV16) was used as a representative virus of the Picornaviridae family of viruses. As shown in FIG. 4 and FIG. 5, 1 μM and 2 μM of Mito-MES alone and 10 μM of DMF alone exhibit little antiviral activity against human rhinovirus type 16 (HRV16) in lung and bronchial epithelial cells. However, the combination of Mito-MES+DMF exhibit synergistic antiviral activity against HRV16. See FIG. 4.

Thus, the antiviral activity of the combination of Mito-MES+DMF against RSV was determined. As shown in FIG. 6, 1 μM Mito-MES+10 μM DMF exhibit a greater fold reduction compared to amount of reduction by 1 μM Mito-MES alone plus the amount of reduction by 10 μM DMF alone. Thus, the combination of Mito-MES and DMF exhibit synergistic antiviral activity against viruses in which they each have little antiviral activity when used alone and also viruses in which they each exhibit significant antiviral activity.

Therefore, in some embodiments, the present invention is directed to a method of preventing, inhibiting, or reducing infection by a virus in a cell or a subject, which comprises administering (a) one or more mitochondrial targeted antioxidants in combination with (b) one or more Nrf2 agonists to the cell or the subject.

In some embodiments, the virus belongs to the Picornaviridae family. In some embodiments, the virus is an Enterovirus. In some embodiments, the virus is an Enterovirus A, an Enterovirus B, an Enterovirus C, an Enterovirus D, an Enterovirus E, an Enterovirus F, an Enterovirus G, an Enterovirus H, an Enterovirus I, an Enterovirus J, an Enterovirus K, an Enterovirus L, a Rhinovirus A, a Rhinovirus B, or a Rhinovirus C. In some embodiments, the virus is an Enterovirus A, an Enterovirus B, an Enterovirus C, an Enterovirus D, a Rhinovirus A, a Rhinovirus B, or a Rhinovirus C. In some embodiments, the virus is a Rhinovirus A, a Rhinovirus B, or a Rhinovirus C. In some embodiments, the virus is a Rhinovirus A. In some embodiments, the virus is human rhinovirus HRV16.

In some embodiments, the virus belongs to the Pneumoviridae family. In some embodiments, the virus is a Metapneumovirus or a Orthopneumovirus. In some embodiments, the virus is a human Metapneumovirus or a human Orthopneumovirus. In some embodiments, the virus is a human respiratory syncytial virus. In some embodiments, the virus is a human metapneumovirus (HMPV), a human respiratory syncytial virus A2 (HRSV-A2), or a human respiratory syncytial virus B1 (HRSV-B1).

### Treatments and Compositions

A plasma concentration of about 2 ng/ml of Mito-MES in a subject is protective against infection by SARS-COV-2 at exposure levels typical of regular and repeated close familial contact. That is, administration of 20 mg/day of Mito-MES, which typically results in a plasma concentration of about 2 ng/ml of Mito-MES, to a subject prevents or inhibits the development of an infection by SARS-COV-2 in the subject over the period the subject is exposed to the virus.

Therefore, in some embodiments, the present invention provides methods of preventing or inhibiting an infection by a virus, e.g., a virus belonging to the Orthomyxoviridae family, in a subject by causing the subject to have a plasma concentration of about 2 ng/ml or higher of a mitochondrial targeted antioxidant, e.g., Mito-MES, during the period the subject is exposed to the virus by administering a therapeutically effective amount of the mitochondrial targeted antioxidant. In some embodiments, the present invention provides methods of preventing or inhibiting a virus, e.g., a virus belonging to the Orthomyxoviridae family, infection in a subject by administering to the subject about 10-20 mg, preferably about 20 mg, of a mitochondrial targeted antioxidant, e.g., Mito-MES, daily for at least about 1, 2, 3, 4, 5, 6, 7, 8, 9, or 10 days, preferably about 5-7 days, post exposure. In some embodiments, administration of the mitochondrial targeted antioxidant begins prior to expected or likely exposure to the virus. In some embodiments, administration of the mitochondrial targeted antioxidant begins at the time of or within about 96 hours, preferably within about 24 hours, more preferably within about 12 hours, of likely or actual exposure to the virus.

In some embodiments, one or more the mitochondrial targeted antioxidants are administered to subjects “in need thereof”. As used herein, subjects “in need of” a the mitochondrial targeted antioxidant include those who are likely to be exposed or have been exposed to a virus and include those who belong to “high-risk” groups (e.g., elderly, those suffering from comorbidities, immunocompromised subjects, those who have not been vaccinated against the given virus, first responders, and health care workers).

As used herein, a “viral disease” refers to a disease caused by infection by a virus. Such “viruses” include those belonging to the Orthomyxoviridae family, the Picornaviridae family, and the Pneumoviridae family of viruses. In some embodiments, the virus is a virus that infects and causes viral disease in humans.

As used herein, a “mitochondrial targeted antioxidant” refers to an antioxidant that scavenges reactive oxygen species in mitochondria (“Mito-ROS”). Mitochondrial targeted antioxidants include mitoquinone (10-(4,5-dimethoxy-2-methyl-3,6-dioxo-1,4-cyclohexadienyl) decyl triphenylphosphonium), mitoquinol (10-(4,5-dimethoxy-2-methyl-3,6-dihydroxy-1,4-cyclohexadienyl) decyl triphenylphosphonium), and their derivatives and salts thereof (e.g., mesylate). Mitochondrial targeted antioxidants also include antioxidants known in the art that scavenge Mito-ROS such as SkQ1 (Mitotech, S.A.), Elamipretide (Stealth BioTherapeutics), Mito-TEMPO (CAS 1569257-94-8), and those disclosed in the following patents and publications: U.S. Pat. Nos. 8,518,915; 9,192,676; 9,328,130; 9,388,156; US20070161609; US20070225255; US20080161267; US20100168198; US20160200749; US20180305328; US20190248816; US20190330249; US20190374558; WO2005019232; WO2006005759; WO2007046729; WO2008145116; WO2015063553; WO2017106803; and WO2018162581, which are herein incorporated by reference. As used herein, “Mito-MES” is used to refer to mitoquinone (10-(4,5-dimethoxy-2-methyl-3,6-dioxo-1,4-cyclohexadienyl) decyl triphenylphosphonium) mesylate and/or mitoquinol (10-(4,5-dimethoxy-2-methyl-3,6-dihydroxy-1,4-cyclohexadienyl) decyl triphenylphosphonium) mesylate.

A derivatives of mitoquinone and mitoquinol refer to compounds having the following structural formula as part of its backbone structure:

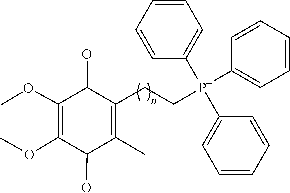

wherein “n” is any number, preferably n is 1-15, more preferably n is 5-10, most preferably n is 9.

As used herein, a “Nrf2 agonist” refers to agonists and activators of nuclear factor erythroid 2-related factor 2 (Nrf2) and the Nrf2 signaling pathway. Exemplary Nrf2 agonists include Antcin C, Baicalein, Butein and phloretin, Carthamus red, Curcumin, Diallyl disulfide, Ellagic acid, Gastrodin, Ginsenoside Rg1, Ginsenoside Rg3, Glycyrrhetinic acid, Hesperidin, Isoorientin, Linalool, Lucidone, Lutein, Lycopene, Mangiferin, Naringenin, Oleanolic acid, Oroxylin A, Oxyresveratrol, Paeoniflorin, Puerarin, Quercetin, Resveratrol, S-Allylcysteine, Salvianolic acid B, Sauchinone, Schisandrin B, Sulforaphane, Tungtungmadic acid, Withaferin A, Alpha-lipoic acid, and Dimethyl fumarate (DMF).

Compositions, including pharmaceutical compositions, comprising one or more mitochondrial targeted antioxidants are contemplated herein.

The term “pharmaceutical composition” refers to a composition suitable for pharmaceutical use in a subject. A composition generally comprises an effective amount of an active agent and a diluent and/or carrier. A pharmaceutical composition generally comprises a therapeutically effective amount of an active agent and a pharmaceutically acceptable carrier. In addition to the one or more mitochondrial targeted antioxidants, pharmaceutical compositions may include one or more supplementary agents. Examples of suitable supplementary agents include anti-inflammatory agents and antiviral agents known in the art.

As used herein, an “effective amount” refers to a dosage or amount sufficient to produce a desired result. The desired result may comprise an objective or subjective change as compared to a control in, for example, in vitro assays, and other laboratory experiments. As used herein, a “therapeutically effective amount” refers to an amount that may be used to treat, prevent, or inhibit a given disease or condition in a subject as compared to a control, such as a placebo. Again, the skilled artisan will appreciate that certain factors may influence the amount required to effectively treat a subject, including the degree of the condition or symptom to be treated, previous treatments, the general health and age of the subject, and the like. Nevertheless, effective amounts and therapeutically effective amounts may be readily determined by methods in the art.

The one or more mitochondrial targeted antioxidants may be administered, preferably in the form of pharmaceutical compositions, to a subject. Preferably the subject is mammalian, more preferably, the subject is human. Preferred pharmaceutical compositions are those comprising at least one mitochondrial targeted antioxidant in a therapeutically effective amount and a pharmaceutically acceptable vehicle. In some embodiments, a therapeutically effective amount of a mitochondrial targeted antioxidant ranges about 0.05 mg/kg to about 15 mg/kg, preferably about 0.2 mg/kg to about 1.5 mg/kg, or more preferably about 0.3 mg/kg to about 0.7 mg/kg body weight. It should be noted that treatment of a subject with a therapeutically effective amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

Pharmaceutical compositions may be formulated for the intended route of delivery, including intravenous, intramuscular, intra peritoneal, subcutaneous, intraocular, intrathecal, intraarticular, intrasynovial, cisternal, intrahepatic, intralesional injection, intracranial injection, infusion, and/or inhaled routes of administration using methods known in the art. Pharmaceutical compositions may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art.

The compositions may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular mitochondrial targeted antioxidant used.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20th ed (2000) Lippincott Williams & Wilkins, Baltimore, MD.

The pharmaceutical compositions may be provided in dosage unit forms. As used herein, a “dosage unit form” refers to physically discrete units suited as unitary dosages for the subject to be treated; each unit containing a predetermined quantity of the one or more mitochondrial targeted antioxidant calculated to produce the desired therapeutic effect in association with the required pharmaceutically acceptable carrier. The specification for the dosage unit forms of the invention are dictated by and directly dependent on the unique characteristics of the given mitochondrial targeted antioxidant and desired therapeutic effect to be achieved, and the limitations inherent in the art of compounding such an active compound for the treatment of individuals.

Toxicity and therapeutic efficacy of mitochondrial targeted antioxidants according to the instant invention and compositions thereof can be determined using cell cultures and/or experimental animals and pharmaceutical procedures in the art. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. Mitochondrial targeted antioxidants which exhibit large therapeutic indices are preferred. While mitochondrial targeted antioxidants that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more mitochondrial targeted antioxidants can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

The following examples are intended to illustrate but not to limit the invention.

### EXAMPLES

### Cells

A546 lung epithelial cells were purchased from American Type Culture Collection (ATCC) (Manassas, VA). Normal human bronchial epithelial cells (NHBE) (#CC-2540) were obtained from Lonza (Basel, Switzerland), and all samples were de-identified. Lonza lung samples were obtained from donors ranging between 30-50 years and represented both males and females. A546, Hela and MDCK cells were maintained at 37° C. and 5% CO₂ in DMEM or MEM supplemented with 10% (v/v) FBS, penicillin (100 units/ml), and streptomycin (100 μg/ml) (1×P/S).

### Reagents

The following materials were purchased from MilliporeSigma (Burlington, MA): DMF (#242926-25G), Dimethyl Sulfoxide (DMSO) (#472301). The following reagents were purchased from Cayman Chemical (Ann Arbor, MI): mitoquinone mesylate (#29317), mitoquinol mesylate (#89950).

### HBEC Air-Liquid Interface Cultures (Upper Airway ALI Cultures)

24-well 6.5 mm transwells with 0.4 μm pore polyester membrane inserts were coated with collagen type I dissolved in cell culture grade water at a ratio of 1:10. 100 μl was added to each transwell and allowed to air dry. HBECs were seeded at 100,000 cells per well directly onto collagen-coated transwells and allowed to grow in the submerged phase of culture for 4-5 days with 500 μl media in the basal chamber and 200 μl media in the apical chamber. ALI cultures were then established and cultured with only 500 μl media in the basal chamber, and cultures were infected with the given virus as indicated. Media was changed every other day and cultures were maintained at 37° C. and 5% CO₂.

### Viral Infection

All viruses were obtained through ATCC. Viruses were passaged once in the indicated cell line and viral stocks were aliquoted and stored at −80° C. Virus titer was measured by median tissue culture infectious dose (TCID₅₀) assay. Cell cultures in 96 well plates and ALI cultures were infected with viral inoculum [Multiplicity of infection (MOI) of 0.5; 100 μl/well] prepared in media. For mock infection, conditioned media (100 μl/well) alone was added.

### Drug Treatments

A concentration of Mito-MES between <2000 nM has been shown to be physiologically relevant, efficacious and non-cytotoxic in human mammalian cells. The antiviral activity of Mito-MES was evaluated in A546 and human bronchial airway (HBEC) air-liquid interface ALI cell cultures. All viral studies were performed in biological triplicate. Cultured cells were incubated separately with Mito-MES. The concentration of DMSO vehicle control was maintained constant at 0.01% v/v for all treatments. Drug effects were measured relative to vehicle controls in vitro. Cell cultures were pretreated for 4 hours (hrs) with the indicated treatments (Mito-MES, DMF,) or vehicle control. The cells were then washed, infected with virus for 2 hrs, the virus was removed, and the treatments were added back. Antiviral agents (oseltamivir for H1N1 and pleconaril for HRV16) were used as an antiviral control.

### RNA Extraction and Real-Time Quantitative Reverse Transcription Polymerase Chain Reaction (RT-qPCR)

The intracellular genomic expression of viral nucleic acid was assessed using primers specific for the given virus and real time PCR. Total RNA was isolated using the RNeasy Mini Kit or Direct-zol RNA Miniprep kit (Zymo Research) and complementary deoxyribonucleic acid (cDNA) was synthesized using oligo deoxythymine (dT) primers and RevertAid first strand cDNA synthesis kit. Quantitative real-time reverse transcription PCR was performed using SYBR Green Master Mix and primers specific for H1N1, RSV and HRV16 as well as glyceraldehyde 3-phosphate dehydrogenase (GAPDH) transcripts. All qRT-PCR reactions were performed using BIO-RAD CFX96 Touch Real-Time PCR Detection System (Bio-Rad Laboratories, Hercules, CA) on 96-well plates. PCR reactions included SYBR Green RT-PCR Master Mix, 10 μM primers and 5 μl of cDNA. Reactions were incubated at 45° C. for 10 min for reverse transcription, 95° C. for 2 min, followed by 40 cycles of 95° C. for 15 seconds (sec) and 60° C. for 60 sec. Gene expression fold change was calculated with the Delta-delta-cycle threshold (DDCt) method. Viral RNA levels were normalized to GADPH as an endogenous control and depicted as fold change over mock infected samples.

### Statistics

Unless noted, error bars in all Figures represent mean and standard error of means (SEM). In the Figures, p-values are presented for comparisons between treatment groups and controls and are denoted by asterisks. To pool cells from different experiments, each measurement was first normalized to the vehicle controls of each experiment. Each experiment contains at least 3 biological replicates (number of wells) and each analysis contains at least 2 independent experiments. For analysis of data that contains more than 2 groups, the Kruskal-Wallis test was performed to compare samples; if these comparisons had a p value less than 0.05 then Mann-Whitney U tests were used to compare statistical difference between 2 groups. P-values less than 0.05 by Kruskal-Wallis or Mann-Whitney were considered significant. All analyses were performed with GraphPad, version 8.0 (GraphPad Holdings, San Diego, CA).

### REFERENCES

The following references are herein incorporated by reference in their entirety with the exception that, should the scope and meaning of a term conflict with a definition explicitly set forth herein, the definition explicitly set forth herein controls:
Bachschmid MM, Schildknecht S, Matsui R, et al. Vascular aging: chronic oxidative stress and impairment of redox signaling-consequences for vascular homeostasis and disease. Ann Med 2013; 45:17-36.
Chen IY, Moriyama M, Chang MF, Ichinohe T. Severe Acute Respiratory Syndrome Coronavirus Viroporin 3a Activates the NLRP3 Inflammasome. Front Microbiol 2019; 10:50.
Cho HY, Imani F, Miller-DeGraff L, et al. Antiviral activity of Nrf2 in a murine model of respiratory syncytial virus disease. Am J Respir Crit Care Med 2009; 179:138-50.
Espinoza JA, Gonzalez PA, Kalergis AM. Modulation of Antiviral Immunity by Heme Oxygenase-1. Am J Pathol 2017; 187:487-93.
Hosakote YM, Liu T, Castro SM, Garofalo RP, Casola A. Respiratory syncytial virus induces oxidative stress by modulating antioxidant enzymes. Am J Respir Cell Mol Biol 2009; 41:348-57.
Kesic MJ, Simmons SO, Bauer R, Jaspers I. Nrf2 expression modifies influenza A entry and replication in nasal epithelial cells. Free Radic Biol Med 2011; 51:444-53.
Komaravelli N, Tian B, Ivanciuc T, et al. Respiratory syncytial virus infection down-regulates antioxidant enzyme expression by triggering deacetylation-proteasomal degradation of Nrf2. Free Radic Biol Med 2015; 88:391-403.
Kosmider B, Messier EM, Janssen WJ, et al. Nrf2 protects human alveolar epithelial cells against injury induced by influenza A virus. Respir Res 2012; 13:43.
Lakatta EG. Arterial and cardiac aging: major shareholders in cardiovascular disease enterprises: Part III: cellular and molecular clues to heart and arterial aging. Circulation 2003; 107:490-7.
Lederman MM, Funderburg NT, Sekaly RP, Klatt NR, Hunt PW. Residual immune dysregulation syndrome in treated HIV infection. Adv Immunol 2013; 119:51-83.
Leng SX, Margolick JB. Understanding frailty, aging, and inflammation in HIV infection. Curr HIV/AIDS Rep 2015; 12:25-32.
Olagnier D, Farahani E, Thyrsted J, et al. SARS-COV2-mediated suppression of NRF2-signaling reveals potent antiviral and anti-inflammatory activity of 4-octyl-itaconate and dimethyl fumarate. Nat Commun 2020; 11:4938.
Schaedler S, Krause J, Himmelsbach K, et al. Hepatitis B virus induces expression of antioxidant response element-regulated genes by activation of Nrf2. J Biol Chem 2010; 285:41074-86.
Seals DR, Jablonski KL, Donato AJ. Aging and vascular endothelial function in humans. Clin Sci 2011; 120:357-75.
Shin HS, Kim Y, Kim G, et al. Immune Responses to Middle East Respiratory Syndrome Coronavirus During the Acute and Convalescent Phases of Human Infection. Clin Infect Dis 2019; 68:984-92.
Simon PF, McCorrister S, Hu P, et al. Highly Pathogenic H5N1 and Novel H7N9 Influenza A Viruses Induce More Profound Proteomic Host Responses than Seasonal and Pandemic H1N1 Strains. J Proteome Res 2015; 14:4511-23.
Yamada Y, Limmon GV, Zheng D, et al. Major shifts in the spatio-temporal distribution of lung antioxidant enzymes during influenza pneumonia. PLOS One 2012; 7:e31494.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”. As used herein, the phrase “consists essentially of” in the context of a given ingredient in a composition, means that the composition may include additional ingredients so long as the additional ingredients do not adversely impact the activity, e.g., biological or pharmaceutical function, of the given ingredient. For example, a composition that “consists essentially of” a mitochondrial targeted antioxidant means that the may comprise additional ingredients so long as the additional ingredients do not adversely affect the functional activity of the mitochondrial targeted antioxidant.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of preventing, inhibiting, or reducing infection by, an inflammatory response caused by infection by, or apoptosis caused by infection by a virus in a cell or a subject or treating the subject for a viral disease caused by infection by the virus, which comprises, consists essentially of, or consists of: (a) administering one or more mitochondrial targeted antioxidants to the cell or the subject, wherein the virus is not a coronavirus or a respiratory syncytial virus (RSV); or (b) administering one or more Nrf2 agonists to the cell or the subject, wherein the virus is not a coronavirus; or (c) administering a combination of one or more mitochondrial targeted antioxidants and one or more Nrf2 agonists to the cell or the subject, wherein the virus is not a coronavirus.

2. (canceled)

3. The method according to claim 1, wherein the one or more mitochondrial targeted antioxidants is mitoquinone mesylate and/or mitoquinol mesylate.

4. The method according to claim 1, wherein the one or more Nrf2 agonists is dimethyl fumarate (DMF).

5. The method according to claim 1, wherein about 0.05 mg/kg to about 15 mg/kg the one or more mitochondrial targeted antioxidants per weight of the subject.

6. The method according to claim 1, wherein about 0.02-8.0 mg/kg, about 0.15-8.0 mg/kg, about 4.0-8.0 mg/kg, about 0.01-4.0 mg/kg, about 0.1-4.0 mg/kg, or about 2.0-4.0 mg/kg of the one or more Nrf2 agonists per weight of the subject.

7. The method according to claim 1, wherein the one or more mitochondrial targeted antioxidants and/or the one or more Nrf2 agonists is administered daily.

8. The method according to claim 1, wherein the one or more mitochondrial targeted antioxidants and/or the one or more Nrf2 agonists is administered orally, subcutaneously, or intravenously.

9. The method according to claim 1, wherein the administration of the one or more mitochondrial targeted antioxidants and/or the one or more Nrf2 agonists is before, during, and/or after the subject was exposed or likely exposed to the virus.

10. The method according to claim 1, wherein the administration of the one or more mitochondrial targeted antioxidants and/or the one or more Nrf2 agonists occurs for at least 1-10 days after the exposure or likely exposure to the virus.

11. The method according to claim 1, wherein the virus
(a) belongs to the Orthomyxoviridae family, preferably the virus is an Alphainfluenzavirus, a Betainfluenzavirus, a Gammainfluenzavirus, a Deltainfluenzavirus, an Isavirus, a Quaranjavirus, or a Thogotovirus, more preferably, the virus is an Influenza A virus, an Influenza B virus, an Influenza C virus, or an Influenza D virus, even more preferably the virus is an Influenza A virus of subtype H1N1, H2N2, H3N2, H5N1, H7N9, H7N7, H1N2, H9N2, H7N2, H7N3, H5N2, H10N7, H10N3, or H5N8;
(b) belongs to the Picornaviridae family, preferably the virus is an Enterovirus (preferably a human Enterovirus), more preferably the virus is an Enterovirus A, an Enterovirus B, an Enterovirus C, an Enterovirus D, a Rhinovirus A, a Rhinovirus B, or a Rhinovirus C virus, even more preferably the virus is a Rhinovirus A, a Rhinovirus B, or a Rhinovirus C virus, and most preferably the virus is a Rhinovirus A virus such as human rhinovirus HRV16, wherein the one or more Nrf2 agonists is administered alone or in combination with the one or more mitochondrial targeted antioxidants; or
(c) belongs to the Pneumoviridae family, preferably the virus is a Metapneumovirus or a Orthopneumovirus, more preferably the virus is a human Metapneumovirus or a human Orthopneumovirus, even more preferably the virus is a human respiratory syncytial virus, and most preferably the virus is a human metapneumovirus (HMPV), a human respiratory syncytial virus A2 (HRSV-A2), or a human respiratory syncytial virus B1 (HRSV-B1), wherein the one or more Nrf2 agonists is administered alone or in combination with the one or more mitochondrial targeted antioxidants.

12. The method according to claim 1, wherein the one or more mitochondrial targeted antioxidants is mitoquinone mesylate and/or mitoquinol mesylate and acts as an antiviral against the virus or prevents, inhibits, or reduces apoptosis caused by infection by the virus, wherein the virus is not a coronavirus or a respiratory syncytial virus (RSV).

13. (canceled)

14. The method according to claim 1, wherein the one or more Nrf2 agonists is dimethyl fumarate (DMF) and acts as an antiviral against the virus or prevents, inhibits, or reduces apoptosis caused by infection by the virus, wherein the virus belongs to the Orthomyxoviridae family, the Picornaviridae family, or the Pneumoviridae family.

15. (canceled)

16. The method according to claim 1, wherein the combination comprises mitoquinone mesylate and/or mitoquinol mesylate plus dimethyl fumarate (DMF), said combination acts as an antiviral against the virus or prevents, inhibits, or reduces apoptosis caused by infection by the virus, wherein the virus is not a coronavirus.

17. A composition comprising a combination of one or more mitochondrial targeted antioxidants plus one or more Nrf2 agonists.

18. The composition according to claim 17, wherein the one or more mitochondrial targeted antioxidants is mitoquinone mesylate and/or mitoquinol mesylate.

19. The composition according to claim 17, wherein the one or more Nrf2 agonists is dimethyl fumarate (DMF).

20. The composition according to claim 17, wherein the one or more mitochondrial targeted antioxidants is mitoquinone mesylate and/or mitoquinol mesylate; and the one or more Nrf2 agonists is dimethyl fumarate (DMF).
